(19) United States
(12) Patent Application Publication
(10) Pub. No.: US 2023/0122280 A1
(43) Pub. Date: Apr. 20, 2023

(54) SYSTEMS AND METHODS FOR PROVIDING VISUAL INDICATORS DURING COLONOSCOPY

(71) Applicant: Covidien LP, Mansfield, MA (US)

(72) Inventor: Mahipal Reddy, Kakinada (IN)

(21) Appl. No.: 17/501,241

(22) Filed: Oct. 14, 2021

(51) Int. Cl. G16H 30/20; G16H 30/40; G16H 50/70; G16H 40/67; A61B 1/00; A61B 1/31; A61B 5/06; G06T 7/00

(52) U.S. Cl. CPC G16H 30/20; A61B 1/0005; A61B 1/31; A61B 5/065; G06T 7/0012; G16H 30/40; G16H 40/67; G16H 50/70; G16H 50/20

(57) ABSTRACT

A system includes a processor, a memory configured to store a digital model of the colon and previous anomalies in the digital model based on previous colonoscopy data and to include instructions stored therein. The instructions, when executed by the processor, cause the system to access a frame image captured by a colonoscope, process the frame image to detect current anomalies and debris, display the frame image on a display, display a first shape at positions of the previous anomalies in the frame image based on correlation between the frame image and the digital model, and display a second shape at positions of the current anomalies in the frame image.

### FIELD

The present technology is generally related to systems and methods for providing visual indicators during colonoscopy, in particular, for overlaying visual indicators in an in-vivo frame image of a colon.

### BACKGROUND

Colorectal cancer is the third leading cause of cancer-related deaths in the United States. Prior to reaching a cancerous stage, colorectal cancer often begins in the form of small polyps, adenomas, or abnormal growths of the colon surface that, when found early, may be easily and safely removed. The preferred screening technique for polyp detection and removal is optical colonoscopy, during which a scope with an attached camera is inserted and guided through the colon to examine the colon wall to find polyps for removal. Since optical colonoscopy requires the attentiveness of medical professionals for identifying polyps, poor diligence or navigational skills for maneuvering the camera lead to missing identification of polyps and anomalies. Thus, real-time display of the changes would enhance identification of polyps or anomalies.

### SUMMARY

To the extent consistent, any or all of the aspects detailed herein may be used in conjunction with any or all of the other aspects detailed herein. Aspects of the present disclosure relate to using a digital model of a colon of a patient based on previously identified polyps or anomalies from a previous colonoscopy, performing in-vivo image processing to identify polyps or anomalies, and displaying one or more visual indicators to bring medical professional’s attention to changes between the previously and currently identified anomalies. Further, previously identified polyps or anomalies may be displayed while current colonoscopy is performed. Thus, displaying changes between the previously and currently identified polyps or anomalies would be helpful for medical professionals to recognize an increase or decrease in size. Furthermore, when polyps or anomalies are obstructed in identification, such obstructions (e.g., debris) should be removed to clearly identify the polyps or anomalies.

In accordance with aspects of the present disclosure, a system includes a processor and a memory configured to store a digital model of a colon and previous anomalies in the digital model based on previous colonoscopy data and to include instructions stored therein. The instructions, when executed by the processor, cause the system to access a frame image captured by a colonoscope, process the frame image to detect current anomalies and debris, display the frame image on a display, display a first shape at positions of the previous anomalies in the frame image based on correlation between the frame image and the digital model, and display a second shape at positions of the current anomalies in the frame image.

In various aspects, the instructions, when executed by the processor, cause the system to receive a position of the colonoscope in the colon from a position sensor. The correlation between the frame image and the digital model is based on the position of the colonoscope.

In various aspects, characteristics of the first shape is displayed differently from characteristics of the second shape. The characteristics include at least one of color, hue, and brightness.

In various aspects, in a case where a current anomaly and a previous anomaly are detected at a same position, the instructions, when executed by the processor, further cause the system to display a directional icon from the first shape to the second shape.

In various aspects, in a case where the debris is detected at a position of a previous anomaly, the instructions, when executed by the processor, further cause the system to display an icon of a water jet near the position.

In various aspects, in a case where the debris is detected at a position where a probability of detecting a current anomaly is higher than a predetermined threshold, the instructions, when executed by the processor, further cause the system to display an icon of a water jet near the position.

In various aspects, in a case where no current anomaly is detected at a position of a previous anomaly, the instructions, when executed by the processor, further cause the system to display an icon of removal over the first shape near the position.

In various aspects, in a case where a previous anomaly is not included in a field of view of the colonoscope, the instructions, when executed by the processor, further cause the system to display a directional icon toward a position of the previous anomaly at a periphery of the frame image.

In accordance with aspects of the present disclosure, a processor-implemented method for colonoscopy includes receiving a frame image of a colon from a colonoscope, processing, by a processor, the frame image to detect current anomalies and debris, displaying the frame image on a display, displaying, on the display, a first shape at positions of previous anomalies, which are based on previous colonoscopy data and saved in a digital model, in the frame image based on correlation between the frame image and the digital model, and displaying, on the display, a second shape at positions of the current anomalies in the frame image.

In various aspects, the processor-implemented method further includes receiving a position of the colonoscope from a position sensor. The correlation between the frame image and the digital model is based on the position of the colonoscope.

In various aspects, characteristics of the first shape is displayed differently from characteristics of the second shape. The characteristics include at least one of color, hue, brightness, and size.

In various aspects, the processor-implemented method further includes, in a case where a current anomaly and a previous anomaly are detected at a same position, displaying, on the display, a directional icon from the first shape to the second shape.

In various aspects, the processor-implemented method further includes, in a case where the debris is detected at a position of a previous anomaly, displaying, on the display, an icon of a water jet at the position.

In various aspects, the processor-implemented method further includes, in a case where the debris is detected at a position where a probability of a current anomaly is higher than a predetermined threshold, displaying, on the display, an icon of a water jet at the position.

In various aspects, the processor-implemented method further includes, in a case where no current anomaly is detected at a position of a previous anomaly, displaying, on the display, an icon of removal at the position.

In various aspects, the processor-implemented method further includes, in a case where a previous anomaly is not included in a field of view of the colonoscope, displaying, on the display, a directional icon toward a position of the previous anomaly at a periphery of the frame image.

In accordance with aspects of the present disclosure, a non-transitory computer readable medium including instructions stored thereon. The instructions, when executed by a computer, cause the computer to perform a method, which includes receiving a frame image of a colon from a colonoscope, processing, by a processor, the frame image to detect current anomalies and debris, displaying the frame image on a display, displaying, on the display, a first shape at positions of previous anomalies, which are based on previous colonoscopy data and saved in a digital model, in the frame image based on correlation between the frame image and the digital model, and displaying, on the display, a second shape at positions of the current anomalies in the frame image.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

The above and other aspects and features of the disclosure will become more apparent in view of the following detailed description when taken in conjunction with the accompanying drawings wherein like reference numerals identify similar or identical elements.

FIG. 1 is a block diagram of a colonoscopy system according to aspects of the present disclosure;

FIG. 2 is a graphical illustration showing of a colon according to aspects of the present disclosure;

FIGS. 3A-3C are graphical illustrations of visual indicators according to aspects of the present disclosure;

FIGS. 4A and 4B are graphical illustrations of debris and a corresponding visual indicator in accordance with aspects of the present disclosure;

FIG. 5 is a graphical illustration of a visual indicator in accordance with aspects of the present disclosure; and

FIG. 6 is a flowchart for displaying visual indicators during a colonoscopy according to aspects of the present disclosure.

### DETAILED DESCRIPTION

The present disclosure relates to systems and methods for performing in-vivo image processing to identify polyps or anomalies, and displaying a visual indicator to bring medical professional’s attention to changes between previously and currently identified polyps and anomalies. Such changes may be displayed as visual indicators over in-vivo frame images. These visual indicators may lead the medical professionals to identify changes between previously and currently identified polyps and anomalies, removal of a previously identified anomaly, a newly identified anomaly, and debris potentially covering previously identified anomalies. Further, the visual indicators may lead the medical professionals to perform further procedure. For example, when debris is placed on a position of a previously identified anomaly, an appropriate visual indicator may be displayed so that the medical professionals is led to push water jet to remove the debris. Furthermore, when a previously identified anomaly is not within the field of view of a colonoscopy, an appropriate visual indicator is displayed so that the medical professionals may know that a previously identified anomaly is not within the field of view of the colonoscopy and know which direction the colonoscopy is to be rotated so that the unseen previously identified anomaly can be within the field of view.

In the following detailed description, specific details are set forth in order to provide a thorough understanding of the disclosure. However, it will be understood by those skilled in the art that the disclosure may be practiced without these specific details. In other instances, well-known methods, procedures, and components have not been described in detail so as not to obscure the present disclosure. Some features or elements described with respect to one system may be combined with features or elements described with respect to other systems. For the sake of clarity, discussion of same or similar features or elements may not be repeated.

Although the disclosure is not limited in this regard, discussions utilizing terms such as, for example, “processing,” “computing,” “calculating,” “determining,” “establishing,” “analyzing,” “checking,” or the like, may refer to operation(s) and/or process(es) of a computer, a computing platform, a computing system, or other electronic computing device, that manipulates and/or transforms data represented as physical (e.g., electronic) quantities within the computer’s registers and/or memories into other data similarly represented as physical quantities within the computer’s registers and/or memories or other information non-transitory storage medium that may store instructions to perform operations and/or processes. Although the disclosure is not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items. Unless explicitly stated, the methods described herein are not constrained to a particular order or sequence. Additionally, some of the described methods or elements thereof can occur or be performed simultaneously, at the same point in time, or concurrently.

A colonoscopy or corresponding procedure as disclosed herein may be extended to cover in-vivo imaging and displaying of portions of gastrointestinal tract (GIT) or may be used for specific use (e.g., for checking the status of a GIT disease, such as Crohn’s disease, or for colon cancer screening).

The terms screen(s), view(s) and display(s) may be used herein interchangeably and may be understood according to the specific context.

The terms “image” and “frame” may each refer to or include the other and may be used interchangeably in the present disclosure to refer to a single capture by an imaging device. For convenience, the term “image” or “frame image” may be used more frequently in the present disclosure and apply to a frame of images.

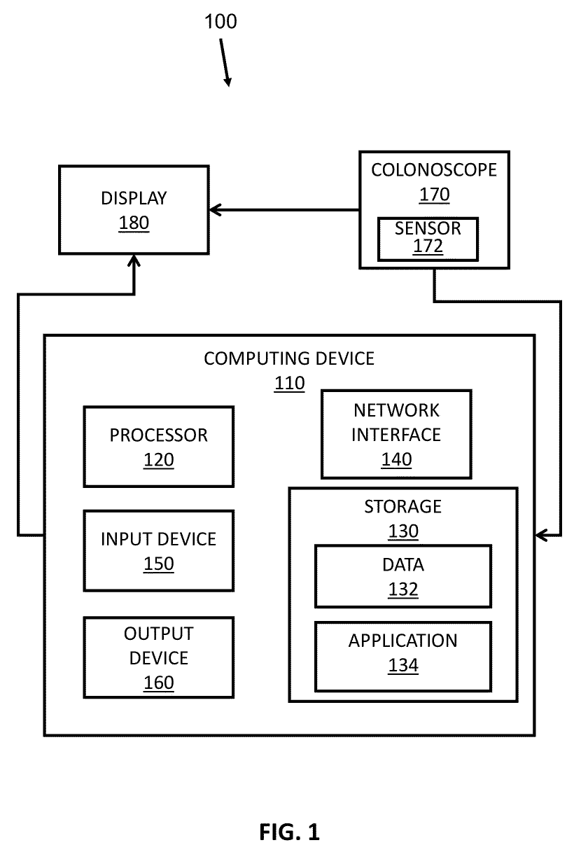

Referring to FIG. 1, an illustration of a colonoscopy system 100 is depicted. The colonoscopy system 100 may be used within humans and other animals. The colonoscopy system 100 may include a computing device 110, a colonoscope 170, and a display 180. The colonoscope 170 may navigate a colon of a human or an animal and capture frame images of the colon. The display 180 may receive the frame images from the colonoscope 170 and displays in-vivo frame images thereon. The colonoscope 170 may provide the in-vivo frame images to the computing device 110, which performs image processing to analyze the frame images. In aspects, the computing device 110 may apply machine learning algorithms to analyze the frame images of the colon. The illustrated components of the computing device 110 and the colonoscope 170 will be described later herein.

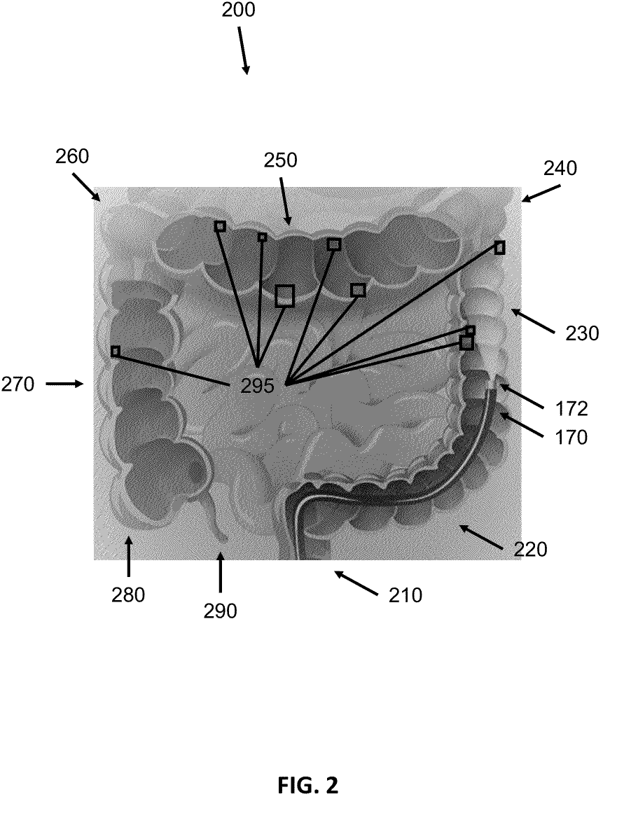

With reference to FIG. 2, an illustration of the colon 200 is shown. The colon 200 absorbs water and any remaining waste material is stored as feces before being removed by defecation. the colon 200 may be divided by, for example, into five anatomical segments: a rectum 210 which is vertically positioned, a sigmoid colon 220 which is shaped as the letter “s” and attached to the rectum 210, a left or descending colon 230 which travels down the left abdomen, a transverse colon 250 which is the longest and most movable part of the colon 200, and a right or ascending colon 270 which lies on the right side of the abdominal cavity. The name sigmoid means S-shaped. The walls of the sigmoid colon 220 are muscular, and contract to increase the pressure inside the colon 200, causing the stool to move into the rectum 210. The sigmoid colon 220 is supplied with blood from several branches of the sigmoid arteries. The rectum 210 is the last section of the colon 200. The rectum 210 holds the formed feces awaiting elimination via defecation.

The transverse colon 250 hangs off the stomach, attached to it by a large fold of peritoneum called the greater omentum. On the posterior side, the transverse colon 250 is connected to the posterior abdominal wall by a mesentery known as the transverse mesocolon.

One function of the descending colon 230 in the digestive system is to store feces that will be emptied into the rectum. The descending colon 230 is also called the distal gut, as it is further along the gastrointestinal tract than the proximal gut. Gut flora is generally very dense in this region.

The colon 200 further includes a left splenic flexure 240 which is the bend portion between the descending colon 230 and the transverse colon 250, and a right splenic flexure 260 which is the bending and connecting portion between the transverse colon 250 and the ascending colon 270. Furthermore, the colon 200 includes a cecum 280 which a pouch connected to the junction of the small and large intestines, and an appendix 290 which is a narrow, finger-shaped pouch that projects out from the colon 200.

The colonoscope 170 may enter into the rectum 210 of the colon 200 via an anus and may travel to the cecum 290. In aspects, the colonoscope 170 may further enter into small intestine as required. As the colon 200 has bent portions at the left and right splenic flexures 240 and 260, between the rectum 210 and the sigmoid colon 220, and any other places in other portions, the colonoscope 170 may rotate, bend, or translate to navigate throughout the colon 200. The colonoscope 170 may include a light source 170 which emits light so that the field of view of the colonoscope 170 may be lighted and the colonoscope 170 may capture frame images of the lighted field of view.

The colonoscope 170 may have a field of view, within which the colonoscope 170 captures frame images. The field of view may be changed based on rotation, bending, or translation of the colonoscope 170.

In aspects, the colonoscope 170 may include a water outlet (now shown), through which a medical professional may inject water in front of the colonoscope 170 within the colon 200. Fecal debris (or simply debris) may obstruct or obscure the field of view of the colonoscope 170. By injecting the water through the water outlet, the debris may be removed from the field of view of the colonoscope 170 and clear frame images may be provided to the display 180 and the computing device 110.

While receiving frame images from the colonoscope 170, the computing device 110 may analyze the frame images, identify anomalies (such as polyps) in the frame images, overlay visual indicators over the frame images based on the identification, and display the overlaid frame images on the display 180.

With reference back to FIG. 1, the computing device 110 may include a processor or controller 120 that may be or include, for example, one or more central processing unit processor(s) (CPU), one or more Graphics Processing Unit(s) (GPU or GPGPU), a chip or any suitable computing or computational device, a storage 130, a network interface 140, an input device 150, and an output device 160. Modules or equipment for collecting or receiving (e.g., a receiver worn on a patient) or displaying or selecting for display (e.g., a workstation) medical images collected by the colonoscope 170 may be or include, or may be executed by, the computing device 110. The network interface 140 of the computing device 110 may allow communications with remote or external devices, e.g., via the Internet or another network, via radio or a suitable network protocol such as file transfer protocol (FTP), Wi-Fi®, local area network (LAN), wide area network (WAN), Bluetooth®, near-field communication (NFC), etc.

The computing device 110 may be operated by an operating system that may be or may include any code segment designed and/or configured to execute machine learning algorithms or tasks involving coordination, scheduling, arbitration, supervising, controlling or otherwise managing operation of the computing device 110, for example, scheduling execution of programs. The memory 130 may be or may include, for example, a random access memory (RAM), a read-only memory (ROM), a dynamic RAM (DRAM), a synchronous DRAM (SD-RAM), a double data rate (DDR) memory chip, a flash memory, a volatile memory, a non-volatile memory, a cache memory, a buffer, a short term memory unit, a long term memory unit, or other suitable memory units or storage units. The memory 130 may be or may include a plurality of possibly different memory units. The memory 130 may store for example, instructions to carry out an application 134 (e.g., executable codes), and/or data 132 such as digital colon model, previously identified anomalies, currently identified anomalies, etc.

The application 134 may include any executable code, e.g., a program, a process, task or script. The application 134 may be executed by the controller 120 possibly under control of the operating system. For example, execution of the application 134 may cause the output device 160 or selection for display of medical images as described herein. In aspects, more than one computing device 110 or components of the computing device 110 may be used for multiple functions described herein. For the various modules and functions described herein, one or more computing devices 110 or components of the computing device 110 may be used. Devices that include components similar or different to those included in the computing device 110 may be used, and may be connected to a network via the network interface 140 and used in the colonoscopy system 100. One or more processors 120 may be configured to carry out methods of the present disclosure by for example executing software, code, or machine learning algorithms. The storage 130 may be or may include, for example, a hard disk drive, a floppy disk drive, a compact disk (CD) drive, a CD-Recordable (CD-R) drive, a universal serial bus (USB) device, solid state drive (SSD), USB drive, RAM drive, or other suitable removable and/or fixed storage unit. The data 132 such as instructions, code, medical images, image streams, a digital model of colon, previously identified anomalies, currently identified anomalies, etc. may be stored in the storage 130 and may be loaded from the storage 130 so that it may be processed by the processor 120. In some aspects, some of the components shown in FIG. 2 may be omitted.

The input device 150 may include for example a mouse, a keyboard, a touch screen pad, or any suitable input device. It will be recognized that any suitable number of input devices may be operatively coupled to the computing device 110. The output device 160 may include one or more monitors, screens, displays, speakers, printers, and/or any other suitable output devices. It will be recognized that any suitable number of output devices may be operatively coupled to the computing device 110 as shown by block 160. Any applicable input/output (I/O) devices may be operatively coupled to the computing device 110 via the network interface 140, for example, a wired or wireless network interface card (NIC), a modem, printer, facsimile machine, a USB device, or external hard drive may be included in the input device 150 and/or the output device 160.

Multiple computing devices 110 including some or all of the components shown in FIG. 1 may be used with the described systems and methods. A cloud platform (e.g., a remote server) including components such as computing device 110 of FIG. 1 may receive procedure data such as images and metadata, processes and generate a study, and may also display the generated study for the physician’s review (e.g., on a web browser executed on a workstation or portable computer). An “on-premises” option may use a workstation or local server of a medical facility to store, process, and display images and/or a study.

According to some aspects of the present disclosure, machine learning systems or machine learning algorithms may be trained by previously captured frame images, which include annotations indicating which portions include polyps or anomalies. By processing a number of frame images, machine learning systems or machine learning algorithms may be able to identify anomalies from in-vivo frame images. In various embodiments, machine learning systems or machine learning algorithms may be trained by supervision or no-supervision.

The computing device 110 may generate a digital model of colon by executing the machine learning system or machine learning algorithms. The digital model may be a three-dimensional (3D) model. In aspects, the digital model may include physical structure of the colon and map previously identified anomalies to the digital model. When a colonoscopy is performed again and anomalies are identified in-vivo, currently identified anomalies may be added or previously identified anomalies may be updated based on currently identified anomalies. In this regard, the colonoscope 170 may include a location sensor 172, which senses a location of the colonoscope 170 within the colon and transmits the sensed location to the computing device 110. In turn, the computing device 110 may map the current position of the colonoscope 170 to a location at the digital model of the colon based on correlation between the colon and the digital model of the colon. The location sensor 172 is exemplary, and other manners of identifying or approximating location are contemplated to be within the scope of the present disclosure.

With reference back to FIG. 2, the colon 200 may be a digital 3D model of the colon and may be displayed on the display 180 to show the current position of the colonoscope 170 within the colon based on the sensed location by the location sensor 172. The digital model 200 may further include previously identified anomalies by rectangular boxes 295 based on one or more previous colonoscopies. The shape of the boxes 295 may be in any form within the digital 3D model 200.

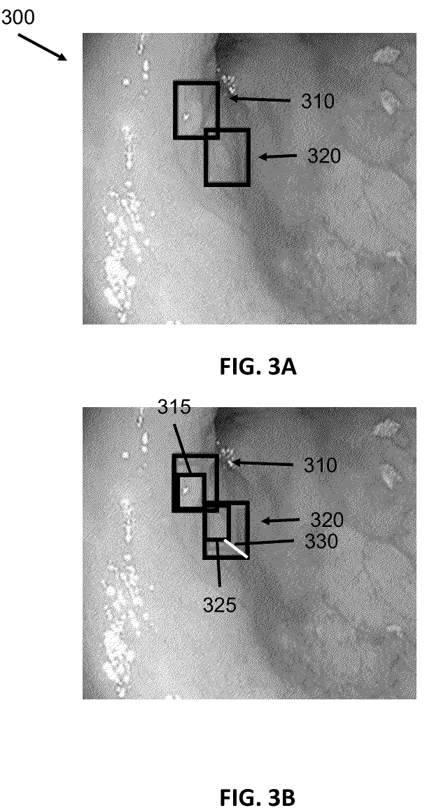

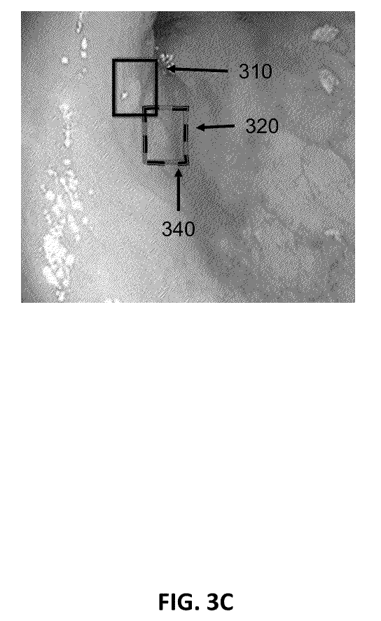

When a new colonoscopy is performed, new anomalies may be found. With reference to FIG. 3A, previously identified anomalies may be indicated as rectangular boxes 310 and 320 within an in-vivo frame image 300 based on the correlation between the digital 3D model 200 and the current location of the colonoscope 170. The machine learning system or machine learning algorithms may identify new anomalies from the frame image 300 at the locations of the previously identified anomalies 310 and 320. With reference to FIG. 3B, as an example, the newly identified anomalies are displayed as smaller rectangular boxes 315 and 325, meaning that the size of the previously identified anomalies 310 and 320 became smaller or decreased. To distinguish the previously identified anomalies 310 and 320 from the currently identified anomalies 315 and 325, the rectangular boxes 310 and 320 may be displayed differently from the rectangular boxes 315 and 325. The color, hue, brightness, and/or shape may be different from each other. For example, with reference to FIG. 3C, one of the previously and currently identified anomalies may be surrounded by a solid line and the other as dotted lines, or one is black and the other is gray. Other changes can be made to one of the previously and currently identified anomalies.

In a case when the display 180 is monochrome or the colors of the previously identified anomalies and the current identified anomalies are same, a directional indicator 330, which is shown in white color, may be also displayed between the previously identified anomalies and the currently identified anomalies. In aspects, the directional indicator 330 may include an arrow head, which indicates a direction to the currently identified anomalies. Even when the color, hue, brightness, and/or shape may be different from each other, the directional indicator 330 may be also displayed with the arrow head to show which is the currently identified anomalies. In a case when the arrow head of the directional indicator 330 shows that the size of the previously identified anomaly has been increased, such will bring the medical professionals’ attention to perform further procedure (e.g., biopsy, removal, etc.).

In an aspect, when an anomaly is not found at a location of a previously identified anomaly, an icon of removal of the previously identified anomaly may be displayed over the previously identified anomaly in the frame image 300 to indicate that the size of the previously identified anomaly has been decreased and disappeared.

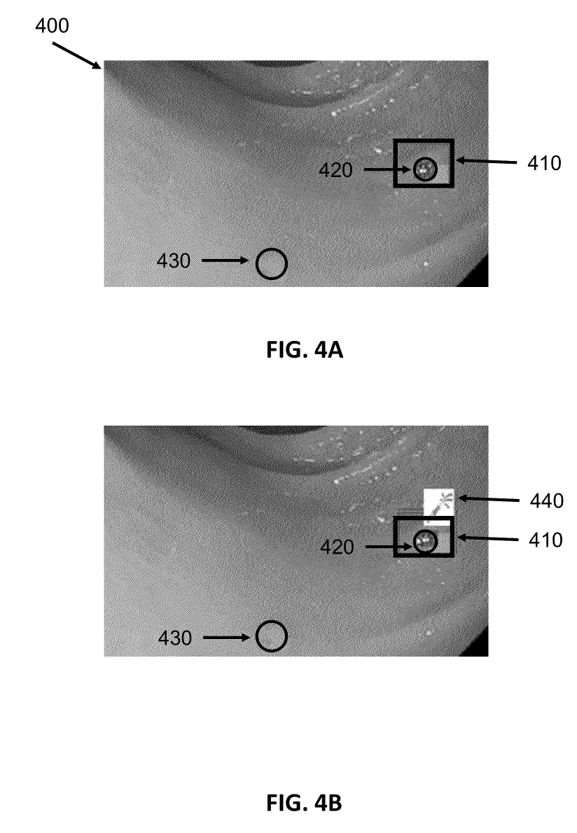

While navigating the colon, debris can cover a portion of the colon. Medical professionals with experience may determine whether to clear the debris to identify anomalies. In a case when debris is positioned at a previously identified anomaly, however, it is beneficial to clear the debris so that increase or decrease in size of the previously identified anomaly can be determined. With reference to FIG. 4A, a rectangular box 410 is displayed at a position of a previously identified anomaly over the in-vivo frame image 400 and debris 420 and 430 are captured in the frame image.

As depicted, the debris 420 is located at the previously identified anomaly 410, while the debris 430 is located at a position where no anomalies had been previously identified. Thus, the machine learning system or machine learning algorithms may display an icon 440 for water jet near the debris 420 to clear the debris 420. The machine learning systems or machine learning algorithms may process frame images captured after clearing the debris 420, identify an anomaly at the location of previously identified anomaly 410, and display an appropriate box (e.g., 315 and 325 in FIG. 3B or 340 in FIG. 3C) for the currently identified anomaly. Then, medical professionals may be able to see an increase or decrease in size between the previously identified anomaly 410 and the currently identified anomaly at the same location. In an aspect, when a new anomaly is identified at or near the debris 430, the water jet icon 440 may be also displayed near the debris 430.

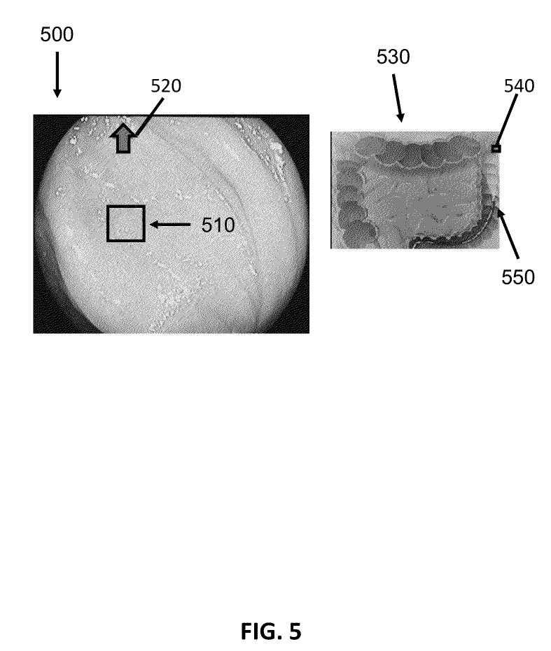

With reference to FIG. 5, a frame image 500 includes one previously identified anomaly 510. A display (e.g., the display 180 of FIG. 1) may show the frame image 500 and a digital 3D model 530 in one screen. The digital 3D model 530 may include a colonoscope 550 to show the current location of the colonoscope 550 within the colon. Further, as shown in FIG. 2, the digital 3D model 530 may also show all previously identified anomalies. For illustrative purposes, the digital 3D model 530 only shows one previously identified anomaly 540, which is not within the field of view of the colonoscope 550, meaning that the frame image 500 does not include the previously identified anomaly 540 therein. In this case, a directional icon 520 may be displayed in the frame image to indicate that the previously identified anomaly 540, which is not displayed in the frame image 500, can be found if the colonoscope 550 is rotated or moved to the direction of the directional icon 540. By displaying the directional icon 520, the previously identified anomalies from previous colonoscopies can be viewed during the current colonoscopy and reviewed based on currently identified anomalies.

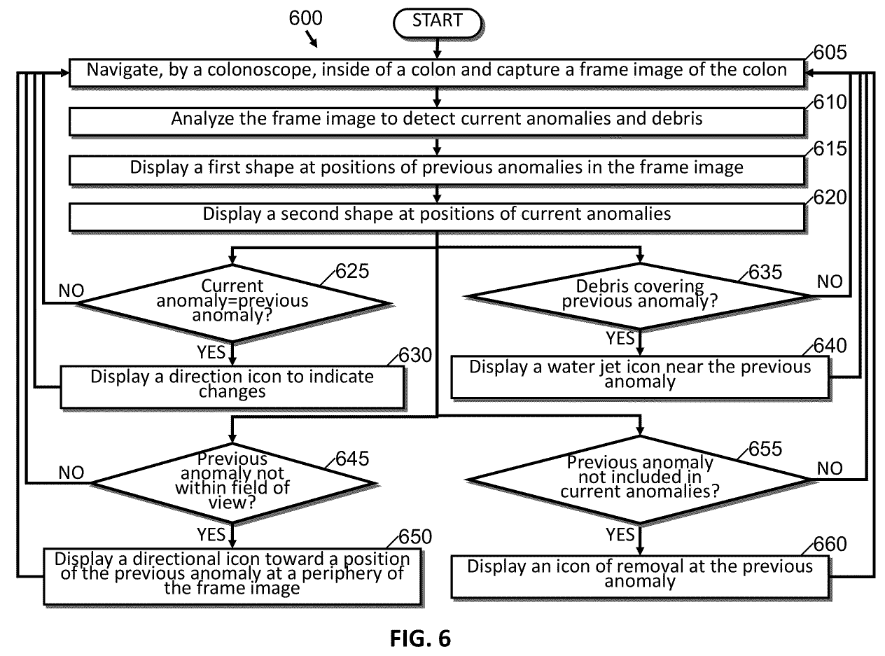

Now referring to FIG. 6, a method 600 for colonoscopy is illustrated according to aspects of the present disclosure. In particular, the method 600 is directed to augmenting visual indicators over a frame image so that medical professionals are able to see differences between previously and currently identified anomalies in the colon.

In step 605, a colonoscope navigates through the colon of a patient and captures frame images of the colon. The colonoscope may have a light source and a water outlet to capture clear frame images.

The captured frame images are analyzed to detect anomalies within the captured frame images in step 610. For analyzing the frame images, machine learning systems and machine learning algorithms may be utilized. In aspects, the machine learning systems and machine learning algorithms may be trained via a supervised or unsupervised manner. For example, previously captured frame images may be provided with annotations of anomalies therewithin and train the machine learning system and machine learning algorithms by reinforcing weights.

The previously identified anomalies may be saved within a digital 3D model in a memory. Based on a location of the colonoscope within the colon and the field of view of the colonoscope, previously identified anomalies may be displayed as a first shape (e.g., a rectangular box or an oval in solid or dotted lines) at the locations thereof within the frame image in step 615.

Also based on the currently identified anomalies within the frame image, a second shape may be displayed within the frame image at the corresponding locations of the currently identified anomalies in step 620. The first and second shapes may be different from each other in size, color, hue, brightness, and/or type of lines.

Visual indicators may be also displayed over the frame image in an augmenting way so that medical professionals may be able to easily identify or recognize differences between the previously and currently identified anomalies.

In step 625, it is determined whether or not the location of a previously identified anomaly is the same as the location of a currently identified anomaly. When it is determined that the location of the previously identified anomaly is the same as the location of the currently identified anomaly, a direction icon (e.g., 330 in FIG. 3B) may be displayed starting from the previously identified anomaly to the currently identified anomaly in step 630. The directional icon may include an arrow head pointing to the currently identified anomaly.

When it is not determined in step 625 that the location of a previously identified anomaly is the same as the location of a currently identified anomaly, the method 600 goes back to step 605.

In step 635, it is determined whether or not debris is covering a previously identified anomaly. When it is determined that debris is covering the previously identified anomaly, it is required to clear the debris to capture a clear frame image. Thus, in step 640, a water jet icon is overlaid near the first shape of the previously identified anomaly. After clearing the debris, steps 605-620 are repeated so that an anomaly can be identified at the location of the previously identified anomaly.

When it is not determined in step 635 that debris is covering a previously identified anomaly, the method 600 goes back to step 605.

In step 645, it is determined whether or not a previously identified anomaly is close to but not within the field of view of the colonoscope. When it is determined that the previously identified anomaly is close to but not within the field of view of the colonoscope, in step 650, a directional icon may be displayed in the periphery of the frame image and close to the location of the previously identified anomaly. The directional icon may have an arrow head directing to the location of the previously identified anomaly. When the colonoscope follows the direction of the arrow head, the previously identified anomaly may be within the field of view of the colonoscope.

When it is not determined in step 645 that the previously identified anomaly is not within the field of view of the colonoscope, the method 600 goes back to step 605.

In step 655, it is determined whether or not a previously identified anomaly is not included in the currently identified anomalies. In other words, it is determined whether or not the second shape is shown without the first shape at one location in the frame image or no shapes are shown at the location of the previously identified anomaly. When it is determined that the previously identified anomaly is not included in the currently identified anomaly, a removal icon may be displayed at the location of the previously identified anomaly in step 660.

When it is not determined in step 655 that the previously identified anomaly is not included in the currently identified anomaly, the method 600 goes back to step 605.

By displaying visual indicators (e.g., the directional icon, the water jet icon, and the removal icon), medical professionals may be able to bring attention so that further actions may be taken, if appropriate. The list of the visual indicators is not limited thereto but can include other types of visual indicators to indicate other differences between the previously and currently identified anomalies.

Steps 625, 635, 645, and 655 may be performed simultaneously or in order. After steps 630, 640, 650, and 660, steps 605-620 may be repeatedly performed until no colonoscopy is required.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A system comprising:
a processor; and
a memory configured to store a digital model of a colon and previous anomalies in the digital model based on previous colonoscopy data and to include instructions stored therein which, when executed by the processor, cause the system to:
access a frame image captured by a colonoscope;
process the frame image to detect current anomalies and debris;
display the frame image on a display;
display a first shape at positions of the previous anomalies in the frame image based on correlation between the frame image and the digital model; and
display a second shape at positions of the current anomalies in the frame image.

2. The system of claim 1, wherein the instructions, when executed by the processor, cause the system to receive a position of the colonoscope in the colon from a position sensor.

3. The system of claim 2, wherein the correlation between the frame image and the digital model is based on the position of the colonoscope.

4. The system of claim 1, wherein characteristics of the first shape is displayed differently from characteristics of the second shape.

5. The system of claim 4, wherein the characteristics include at least one of color, hue, and brightness.

6. The system of claim 1, wherein, in a case where a current anomaly and a previous anomaly are detected at a same position, the instructions, when executed by the processor, further cause the system to:
display a directional icon from the first shape to the second shape.

7. The system of claim 1, wherein, in a case where the debris is detected at a position of a previous anomaly, the instructions, when executed by the processor, further cause the system to:
display an icon of a water jet near the position.

8. The system of claim 1, wherein, in a case where the debris is detected at a position where a probability of detecting a current anomaly is higher than a predetermined threshold, the instructions, when executed by the processor, further cause the system to:
display an icon of a water jet near the position.

9. The system of claim 1, wherein, in a case where no current anomaly is detected at a position of a previous anomaly, the instructions, when executed by the processor, further cause the system to:
display an icon of removal over the first shape near the position.

10. The system of claim 1, wherein, in a case where a previous anomaly is not included in a field of view of the colonoscope, the instructions, when executed by the processor, further cause the system to:
display a directional icon toward a position of the previous anomaly at a periphery of the frame image.

11. A processor-implemented method for colonoscopy, the processor-implemented method comprising:
receiving a frame image of a colon from a colonoscope;
processing, by a processor, the frame image to detect current anomalies and debris;
displaying the frame image on a display;
displaying, on the display, a first shape at positions of previous anomalies, which are based on previous colonoscopy data and saved in a digital model, in the frame image based on correlation between the frame image and the digital model; and
displaying, on the display, a second shape at positions of the current anomalies in the frame image.

12. The processor-implemented method of claim 11, further comprising:
receiving a position of the colonoscope from a position sensor.

13. The processor-implemented method of claim 12, wherein the correlation between the frame image and the digital model is based on the position of the colonoscope.

14. The processor-implemented method of claim 11, wherein characteristics of the first shape is displayed differently from characteristics of the second shape.

15. The processor-implemented method of claim 14, wherein the characteristics include at least one of color, hue, brightness, and size.

16. The processor-implemented method of claim 11, further comprising: in a case where a current anomaly and a previous anomaly are detected at a same position,
displaying, on the display, a directional icon from the first shape to the second shape.

17. The processor-implemented method of claim 11, further comprising: in a case where the debris is detected at a position of a previous anomaly,
displaying, on the display, an icon of a water jet at the position.

18. The processor-implemented method of claim 11, further comprising: in a case where the debris is detected at a position where a probability of a current anomaly is higher than a predetermined threshold,
displaying, on the display, an icon of a water jet at the position.

19. The processor-implemented method of claim 11, further comprising: in a case where no current anomaly is detected at a position of a previous anomaly,
displaying, on the display, an icon of removal at the position.

20. The processor-implemented method of claim 11, further comprising: in a case where a previous anomaly is not included in a field of view of the colonoscope,
displaying, on the display, a directional icon toward a position of the previous anomaly at a periphery of the frame image.

21. A non-transitory computer readable medium including instructions stored thereon which, when executed by a computer, cause the computer to perform a method comprising:
receiving a frame image of a colon from a colonoscope;
processing the frame image to detect current anomalies and debris;
displaying the frame image on a display;
displaying a first shape at positions of previous anomalies, which are based on previous colonoscopy data and saved in a digital model, in the frame image based on correlation between the frame image and the digital model; and
displaying a second shape at positions of the current anomalies in the frame image.
